(19) United States
(12) Patent Application Publication
(10) Pub. No.: US 2008/0243115 A1
(43) Pub. Date: Oct. 2, 2008

(54) Catheter actuator

(76) Inventor: David Ogle, Cowan (AU)

Correspondence Address: MORRISON & FOERSTER LLP, 755 PAGE MILL RD, PALO ALTO, CA, 94304-1018, US

(21) Appl. No.: 11/903,412

(22) Filed: Sep. 20, 2007

Related U.S. Application Data
(60) Provisional application No. 60/846,561, filed on Sep. 21, 2006.

(51) Int. Cl. A61B 18/14

(52) U.S. Cl. 606/41

(57) ABSTRACT

A catheter actuator 10 includes at least two carriers 12, 14 displaceably arranged relative to each other. A first catheter sheath component of a catheter sheath assembly is connectable to a first carrier 12 and a second catheter sheath component of the catheter sheath assembly is connectable to a second carrier 14. A control mechanism 22 is carried, in use, by a catheter handle and is associated with the carriers 12, 14 to effect relative displacement between the carriers 12, 14 to cause displacement of a distal end of at least the first catheter sheath component between a non-deployed position and a deployed position.

### CROSS-REFERENCE TO RELATED APPLICATIONS

This application claims the benefit of U.S. Provisional Application No. 60/846,561, filed on Sep. 21, 2006, the disclosure of which is incorporated herein by reference in its entirety.

### FIELD

This invention relates, generally, to a catheter and, more particularly, to a catheter actuator, to a catheter handle assembly including the actuator and to a catheter assembly.

### BACKGROUND

In the field of heat treatment of tissue, it is desirable if the device heating the tissue is in contact only with the tissue being treated and not surrounding tissue or bodily fluids. This minimises the power required to heat the tissue and also minimises unnecessary damage to other tissue, structures or fluid.

In addition, it is often necessary to overcome tissue irregularities at a site in a patient's body being heat treated. An example where a site in a patient's body is subjected to heat treatment is in the treatment of heart arrhythmias where tissue is ablated in an effort to cure the arrhythmia. The tissue is ablated to create a lesion to block the electrical impulses causing the arrhythmia. To ensure that a lesion of adequate depth is formed, it is desirable that the ablating electrode make good contact with the tissue. Other examples of the use of heat treatment at a site in a patient's body include treatment of Parkinson's disease, tumour ablation, endometriosis and pain management.

Further, a lesion formed by a burn between two spaced electrodes can be more effective than a lesion created between a single electrode and a back plate. The reason for this is that a longer, shallower lesion has a greater likelihood of ablating the affected tissue than a shorter, deeper lesion.

It would be desirable to provide a simple to use actuator to enable a clinician to deploy the electrodes at a distal end of a catheter to enable wide area ablation to be effected.

### SUMMARY

According to a first aspect of the invention, there is provided a catheter actuator which includes

at least two carriers displaceably arranged relative to each other, a first catheter sheath component of a catheter sheath assembly being connectable to a first carrier and a second catheter sheath component of the catheter sheath assembly being connectable to a second carrier; and

a control mechanism carried, in use, by a catheter handle and associated with the carriers to effect relative displacement between the carriers to cause displacement of a distal end of at least the first catheter sheath component between a non-deployed position and a deployed position.

The distal end of the first catheter sheath component, which may be an electrode sheath of a catheter sheath assembly, may define a plurality of petals or leaves which, in their non-deployed position or configuration, extend parallel to a longitudinal axis of the electrode sheath. In the deployed position or configuration of the distal end of the electrode sheath, the leaves or petals may project radially outwardly relative to the longitudinal axis of the electrode sheath to expose at least one electrode carried on operatively inner surface of the leaves of the electrode sheath.

The second catheter sheath component may be a sleeve of the catheter sheath assembly which is co-axially arranged about the electrode sheath. By moving the sleeve in a proximal direction relative to the electrode sheath an outward force is imparted to the leaves of the electrode sheath to cause them to move to their deployed position and vice versa. The sleeve may have leaves or petals arranged outwardly of, and in register with, those of the electrode sheath.

The first carrier and the second carrier may be arranged to be displaceable relative to a further component of the catheter, at least a part of which is fast with a body of the catheter handle. The further component may be a stylet of the catheter assembly. More particularly, the stylet may be a steering shaft of the catheter assembly.

The first carrier may comprise a body to which the first catheter sheath component is connectable, the body defining a bore. The second carrier may comprise a tubular member slidably received in the bore of the body of the first carrier with the second catheter sheath component being connectable to a distal end of the tubular member.

The second carrier may include a follower which cooperates with the control mechanism for controlling relative displacement between the first carrier and the second carrier, the follower projecting through an elongate opening defined in the body of the first carrier.

The control mechanism may include a movement control member mounted, in use, fast with the catheter handle, the movement control member having a guide arrangement for guiding relative displacement between the first carrier and the second carrier. Further, the control mechanism may include an actuator arm carried by the first carrier and which engages the movement control member, the actuator arm defining a receiving formation in which the follower of the second carrier is received. The actuator arm may be pivotally mounted on the first carrier to be pivotable about a pivot axis.

The actuator arm may carry a cooperating formation which cooperates with the guide arrangement of the movement control member, the cooperating formation being arranged on one side of the pivot axis with the receiving formation being arranged on an opposed side of the pivot axis. The guide arrangement may be a shaped, more particularly a cranked, guide slot defined in the movement control member, the cooperating formation of the actuator arm being a second follower received in the guide slot.

According to a second aspect of the invention, there is provided a catheter handle assembly which includes

a handle body; and

a catheter actuator, as described above, carried by the handle body.

The handle body may define a mounting arrangement for mounting the at least part of the further component of the catheter. A guide tube arrangement may be carried within the handle body for guiding the further component.

The assembly may include a catheter sheath projection arrangement carried on a distal end of the handle body. The handle body may be elongate and in which the catheter sheath projection arrangement is axially displaceably arranged on the distal end of the handle body.

The catheter actuator may be arranged within the catheter sheath projection arrangement. The movement control member of the catheter actuator may be arranged in a fixed position relative to the catheter sheath projection arrangement in a bore at the distal end of the handle body. The movement control member may be mounted on a distal part of a steering control mechanism of the catheter handle assembly and the catheter sheath projection arrangement may be axially displaceably arranged on the distal end of the handle body. Further, the body of the first carrier of the catheter actuator may be fast with the catheter sheath projection arrangement.

The invention extends also to a catheter assembly which includes

a catheter handle assembly as described above;

a stylet received in the handle body of the catheter handle assembly to project through a distal end of the handle body; and

a catheter sheath assembly mounted on, and extending distally from, the catheter handle assembly.

The stylet may be a steering shaft received in a lumen of the catheter sheath assembly. The steering shaft may have a first member fixed within the handle body and a second member axially displaceably arranged relative to the first member.

The first member and the second member may be secured together at a distal region of the steering shaft and one of the first and second members may define a bend enhancing region so that, when there is axial displacement of the first member and the second member relative to each other, bending at the distal region of the steering shaft results.

An electrode may be carried on a distal end of the steering shaft.

The catheter actuator may be configured to displace the first catheter sheath component and the second catheter sheath component by a first amount relative to one another and the catheter sheath assembly, comprising the combination of the first catheter sheath component and the second catheter sheath component, by a second, greater amount relative to the steering shaft so that, when the first catheter sheath component is in its deployed position, the electrode of the steering shaft is operatively positioned relative to the deployed first catheter sheath component.

As described above, when the first catheter sheath component, i.e. the electrode sheath, is in its deployed position, its petals extend substantially radially outwardly. Thus, “operatively positioned” means that the electrode of the steering shaft lies substantially in the same plane as the electrodes of the leaves of the electrode sheath.

When the distal end of the at least first catheter sheath component is in its non-deployed configuration, the distal end of the at least first catheter sheath component may lie proximally of the electrode of the steering shaft.

### BRIEF DESCRIPTION OF THE DRAWINGS

FIG. 1 shows a schematic, side view of a catheter actuator, in accordance with an embodiment of the invention, the actuator being in a first position;

FIG. 2 shows a schematic, side view of the actuator in a second position;

FIG. 3 shows a three dimensional view of the actuator in its first position;

FIG. 4 shows a three dimensional view of the actuator in its second position;

FIG. 5 shows a schematic, sectional side view of a distal part of a catheter handle assembly, in accordance with a further embodiment of the invention, with a actuator of the assembly in a first position;

FIG. 6 shows a schematic, sectional side view of the distal part of the catheter handle assembly with the actuator in a second position;

FIG. 7 shows a three dimensional view of a catheter handle assembly;

FIG. 8 shows a schematic, sectional side view of a distal part of a catheter sheath assembly of a catheter assembly in accordance with a further embodiment of the invention with the catheter sheath assembly being in a first, non-deployed configuration; and

FIG. 9 shows a schematic, sectional side view of the distal part of the catheter sheath assembly in a second, deployed configuration.

### DETAILED DESCRIPTION

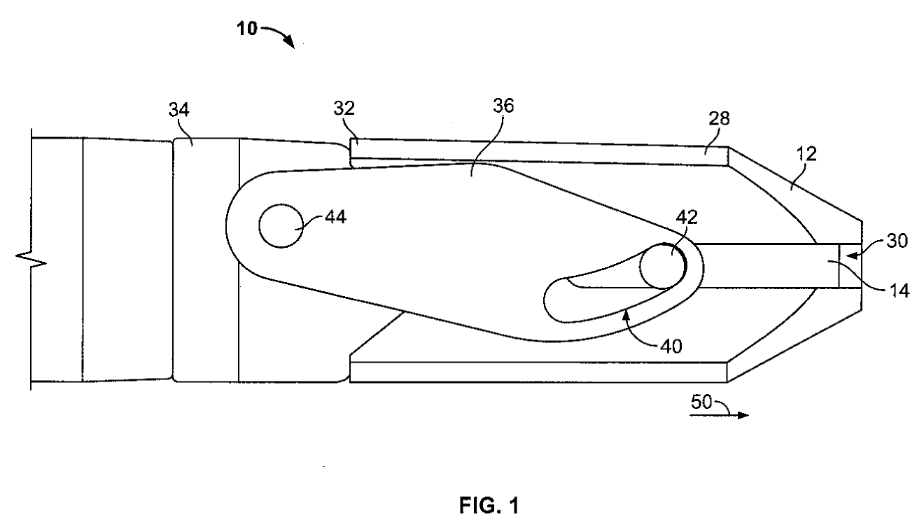

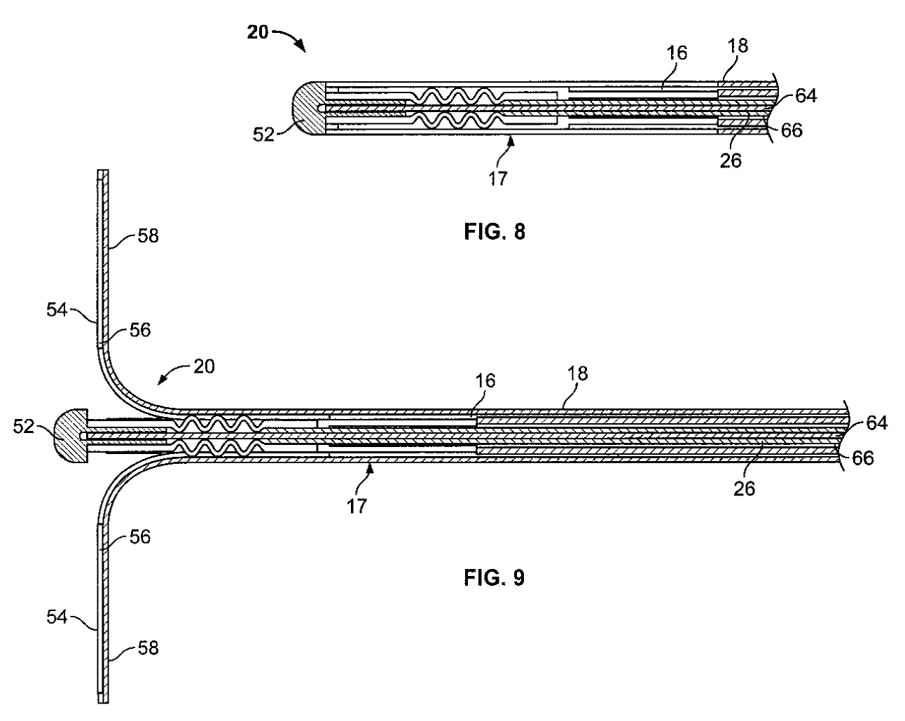

Referring initially to FIGS. 1 to 4 of the drawings, a catheter actuator is illustrated and is designated generally by the reference numeral 10. The actuator 10 includes a first carrier 12 and a second carrier 14 displaceably arranged relative to each other. A first catheter sheath component in the form of an electrode sheath 16 of a catheter sheath assembly 17 (FIGS. 8 and 9) is securable to a distal end of the first carrier 12 and a second catheter sheath component, in the form of a sleeve 18 (FIGS. 8 and 9) of the catheter sheath assembly 17 is securable to a distal end of the second carrier 14.

The catheter sheath assembly 17 has a distal end 20 which is displaceable between a non-deployed configuration as shown in FIG. 8 of the drawings and a deployed configuration as shown in FIG. 9 of the drawings. The movement of the distal end 20 of the catheter sheath assembly 17 between its deployed and non-deployed configurations is controlled by relative movement of the carriers 12 and 14, as will be described in greater detail below.

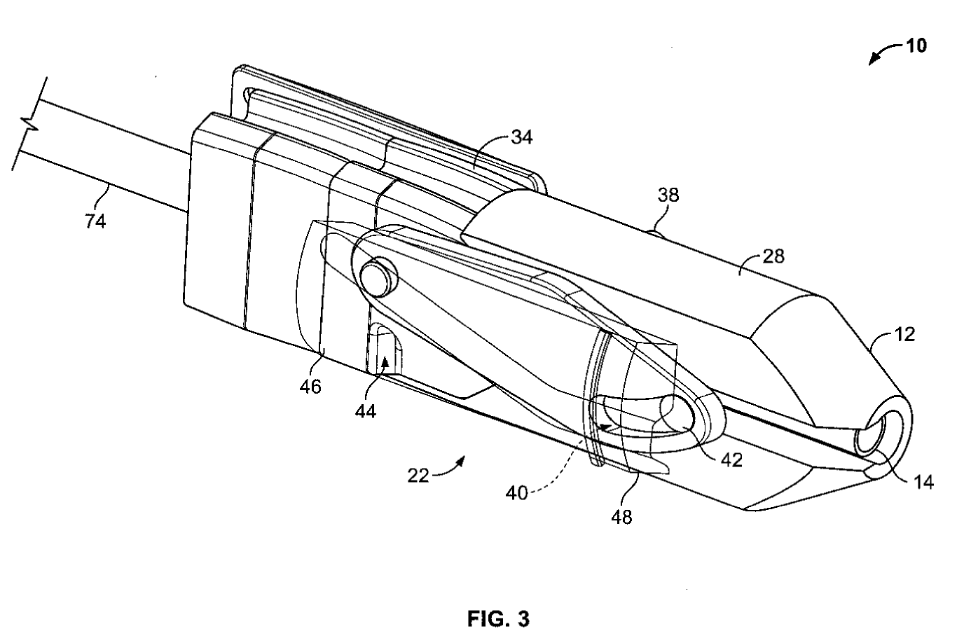

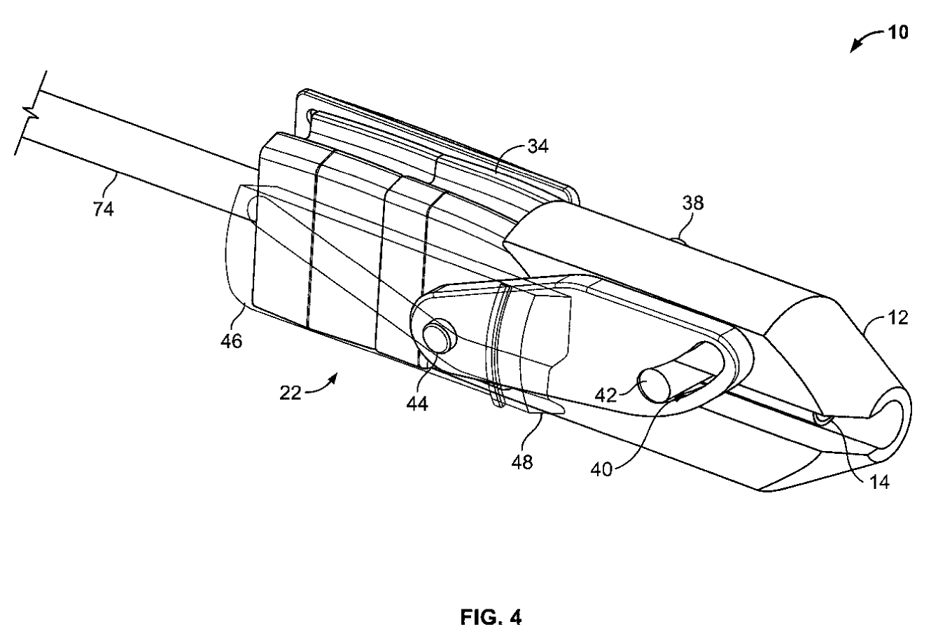

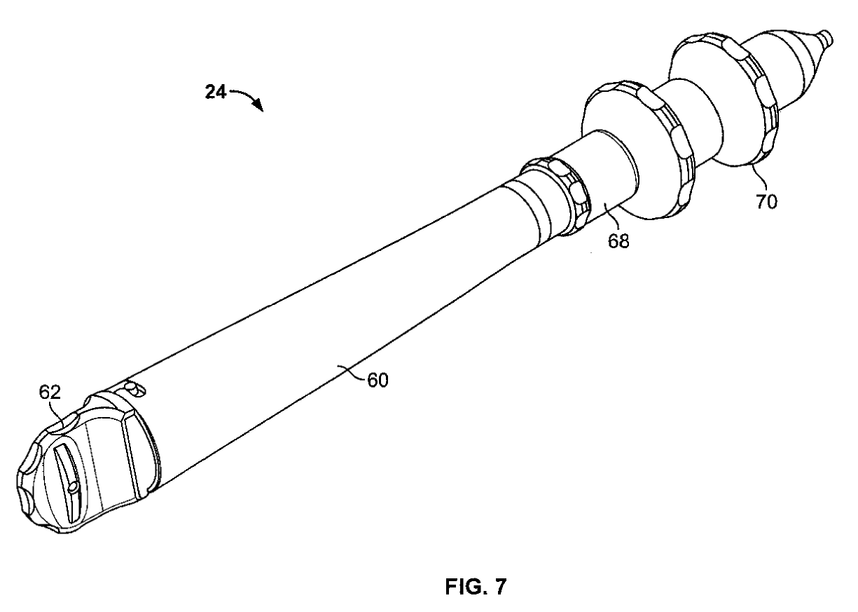

The actuator 10 includes a control mechanism 22 (FIGS. 3 and 4) carried, in use, by a catheter handle 24 (FIG. 7). The control mechanism 22 effects relative displacement between the carriers 12 and 14 to cause displacement of the distal end 20 of the catheter sheath assembly 17 between the non-deployed configuration and the deployed configuration.

Both the first carrier 12 and the second carrier 14 are, in turn, arranged in the catheter handle 24 so as to be displaceable relative to a further component, being a steering shaft 26 (FIGS. 8 and 9) of a catheter assembly.

The carrier 12 is in the form of a body 28 defining a bore 30. A proximal end 32 of the body 28 is shaped to accommodate a boss 34, the body 28 being fast with the boss 34. The boss 34 is used to support a part of the electrode sheath 16 functioning as a cable for connection to a source of RF energy. This part of the electrode sheath 16 is omitted from the drawings for the sake of clarity. The boss 34 also supports an irrigation conduit (not shown) of the catheter assembly, where applicable. The electrode sheath 16 is secured to the boss 34.

The sleeve 18, in turn, is secured to a distal end of the second carrier 14. It will be appreciated that the electrode sheath 16 passes through the second carrier 14 to be secured to the boss 34.

The actuator 10 includes a control mechanism 22 (FIGS. 3 and 4). The control mechanism 22 comprises an actuator arm 36 pivotally mounted on the body 28 of the first carrier 12. The actuator arm 36 is pivotally mounted to the body 28 via a pivot pin 38 (FIGS. 3 and 4) to define a pivot axis about which the actuator arm 36 pivots, in use.

An arcuate slot 40 is defined in a distal portion of the actuator arm 36, distally of the pivot pin 38. A follower 42, which is fast with, and projects radially outwardly from, the tubular member of the second carrier 14, is received in the arcuate slot 40.

A second follower 44 is arranged on the actuator arm 36 proximally of the pivot pin 38. The control mechanism 22 includes a movement control member in the form of a plate 46 (FIGS. 3 and 4) which, in use, is fast with a distal part of the handle body 24. The plate 46 defines a shaped slot 48. More particularly, the slot 48 is cranked and the follower 44 is received in the slot 48. The shape of the slot 48 dictates the manner in which the catheter sheath assembly 17 moves from its non-deployed configuration to its deployed configuration and vice versa. Further, it will be appreciated that, instead of the plate and slot, the movement control member could adopt other configurations which control the deployment of the distal end 20 of the catheter sheath assembly 17. For example, the movement control member could be a fixed cam having a predefined cam surface with at least the follower 44 of the actuator arm 36 being biased to follow the cam surface of the cam.

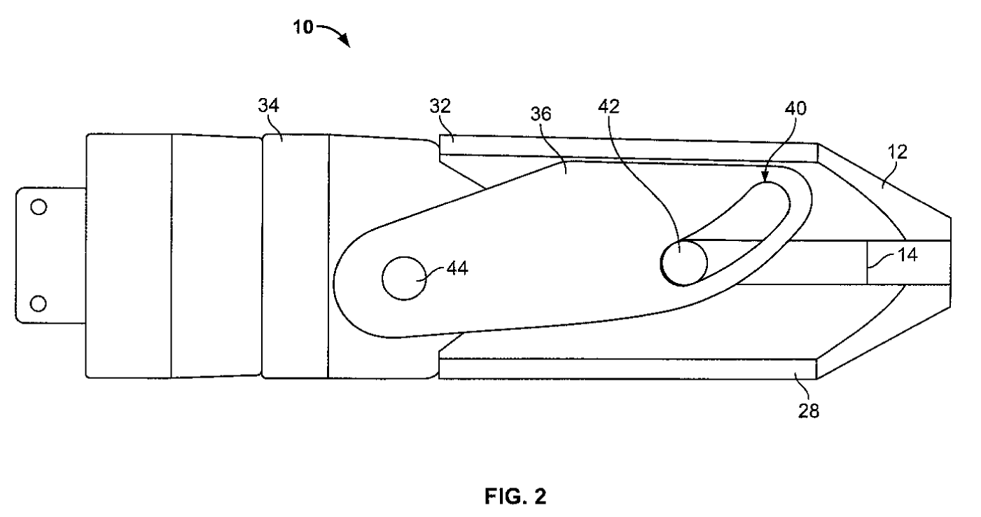

The arrangement is such that, when the first carrier 12 is displaced in a direction of arrow 50 (FIG. 1), the first carrier 12 moves by a predetermined amount relative to the second carrier 14. The amount by which the first carrier 12 moves relative to the second carrier 14 is shown by the relative positions of the carriers 12 and 14 in FIGS. 1 and 2 of the drawings and, similarly, in FIGS. 3 and 4 of the drawings.

The first carrier 12 and the second carrier 14 both move in the direction of the arrow 50 relative to the steering shaft 26 (FIGS. 8 and 9).

Referring again to FIGS. 8 and 9, it is to be noted that an electrode 52 is carried on a distal end of the steering shaft 26. Substantially planar electrodes 54 are carried on operatively inner surfaces of petal portions 56 at a distal end of the electrode sheath 16 of the catheter sheath assembly 17. The sleeve 18 of the catheter sheath assembly 17 has similar leaves 58 arranged in register with the petals 56 of the electrode sheath 16. Relative movement between the electrode sheath 16 and the sleeve 18 causes the petals 56 of the electrode sheath 16 and the leaves 58 of the sleeve 18 to move from the position shown in FIG. 8 of the drawings to the position shown in FIG. 9 of the drawings. More particularly, this occurs when the sleeve 18 is displaced proximally relative to the electrode sheath 16.

However, only approximately 3 mm of relative movement between the electrode sheath 16 and the sleeve 18 is required to effect movement of the petals 56 of the electrode sheath 16 from the non-deployed configuration of FIG. 8 to the deployed configuration of FIG. 9. Each petal 56 is approximately 15 mm in length. In order to bring the electrode 52 of the steering shaft 26 more or less into the same plane as the petals 56 in their deployed configuration, approximately 11 mm of movement is required between the steering shaft 26 and the catheter sheath assembly 17. This is effected by moving the combination of the first carrier 12 and the second carrier 14 in the direction of the arrow 50 relative to the steering shaft 26.

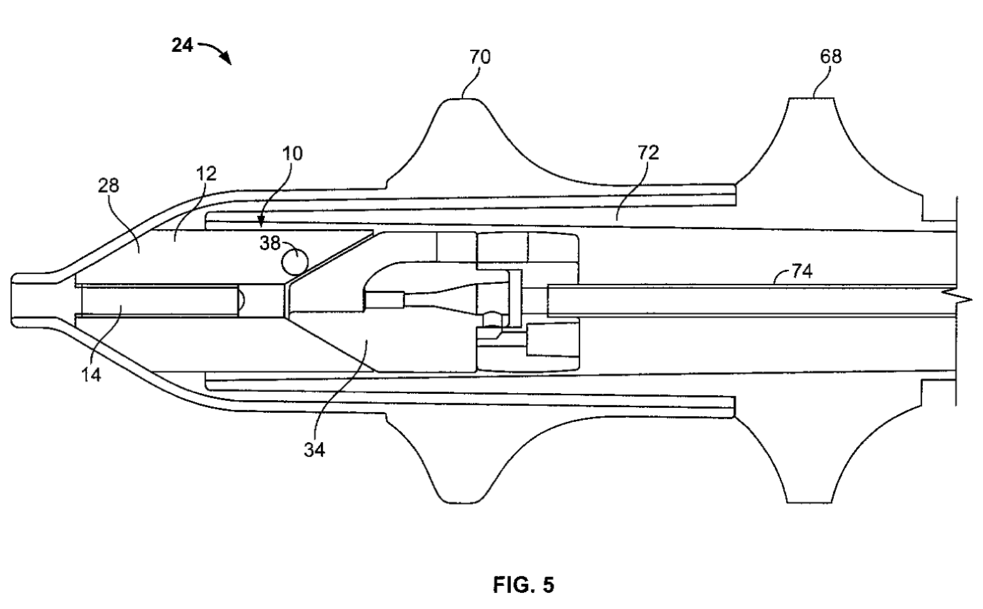

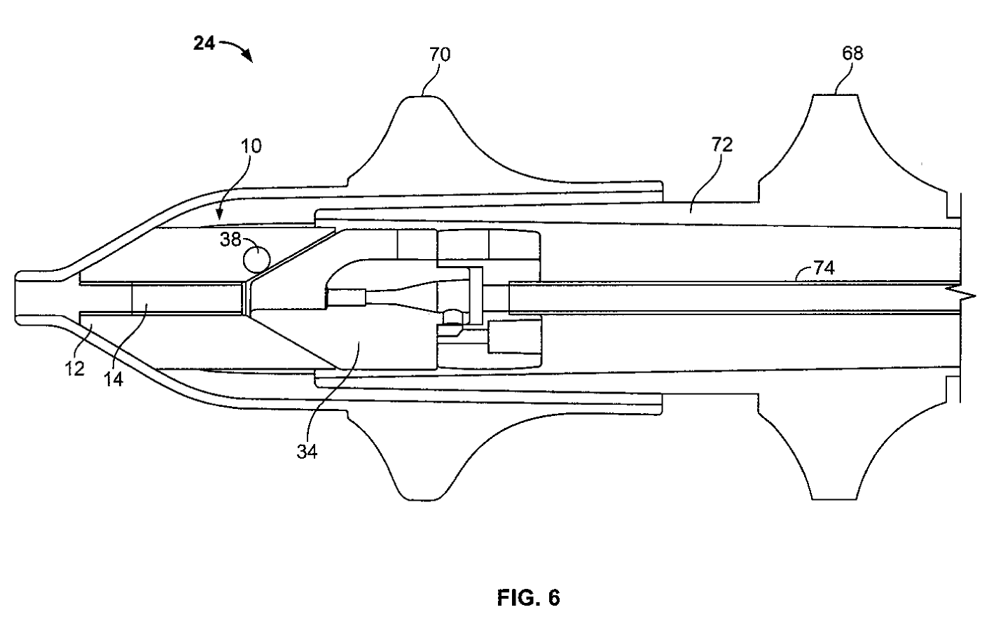

This will be described more clearly below with reference to FIGS. 5 and 6 which show a distal part of the catheter handle 24. The catheter handle 24 includes a handle body 60 (FIG. 7). A control knob 62 is mounted at a proximal end of the handle body. The control knob 62 mounts a first tubular member 64 (FIGS. 8 and 9) of the steering shaft 26. A second tubular member 66 of the steering shaft 26 is co-axially arranged about the first tubular member 64 and is fast with a steering control mechanism 68 carried at a distal end of the handle body 24.

A catheter sheath projection arrangement 70 is carried, in turn, on a distal part of the steering control mechanism 68. Thus, as shown in greater detail in FIGS. 5 and 6 of the drawings, a distal part of the steering control mechanism 68 defines a tubular, axially extending part 72 on which the catheter sheath projection arrangement 70 is carried. The body 28 of the first carrier 12 is fast with the catheter sheath arrangement 70. The handle body 24 carries a guide tube arrangement 74 for guiding the steering shaft 26 through the handle body 24.

In order to deploy the petals 56 of the electrode sheath 16, the catheter sheath projection arrangement 70 is moved from the position shown in FIG. 5 of the drawings to the position shown in FIG. 6 of the drawings. When this occurs, the control mechanism 22 of the actuator 10 causes relative displacement between the carriers 12 and 14 to a lesser extent than the displacement of the catheter sheath projection arrangement 70 relative to the steering control mechanism 68. Thus, while the catheter sheath projection arrangement 70 may move approximately 15 mm relative to the steering control mechanism 68 and, consequently, the first carrier 12 moves by the same amount relative to the steering shaft 26, the carrier 14 moves proximally relative to the carrier 12 by a substantially smaller amount, i.e. by about 3 to 4 mm to cause the distal end 20 of the catheter sheath assembly 17 to move from its non-deployed position shown in FIG. 8 of the drawings to its deployed position shown in FIG. 9 of the drawings.

Once the ablation procedure has been completed, the procedure is reversed and the catheter sheath projection arrangement 70 is moved from the position shown in FIG. 6 of the drawings to the position shown in FIG. 5 of the drawings. Once again, relative movement of about 15 mm between the steering control mechanism 68 and the catheter sheath projection arrangement 70 translates into relative movement between the carriers 12 and 14 of some 3 to 4 mm to move the petals 56 of the catheter sheath assembly 17 back to the non-deployed configuration as shown in FIG. 8 of the drawings.

It is a particular advantage of the invention that a catheter actuator is provided which facilitates deployment of a distal end of the catheter sheath assembly in a simple manner using movements with which a clinician would be familiar. The movement of the catheter sheath projection arrangement 70 is an operation with which the clinician would be familiar arising from using a catheter handle as described in the applicant's co-pending International Patent Application No. PCT/AU2006/000266 dated 1 Mar. 2006, entitled “A catheter handle and a catheter assembly including such a handle”. A catheter handle of the type described in that patent application provides a benefit in that the clinician has the option of displacing the distal end of an electrode sheath relative to the steering shaft in circumstances where such an action is desirable, eg to obtain access to a difficult to reach site in the patient's body. Thus, by means of a similar movement, the clinician is able to deploy the petals 56 of the catheter sheath 16. Hence, it is a movement which would come naturally to the clinician and facilitates ease of use of the catheter assembly.

Additionally, it is an advantage of the invention that a catheter actuator is provided which enables the catheter sheath assembly to be manipulated between its deployed and non-deployed positions in a one-handed operation.

In addition, the arrangement of the actuator 10 is such that the size of the handle 24 is not increased to any significant extent.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A catheter actuator comprising:
at least two carriers displaceably arranged relative to each other, a first catheter sheath component of a catheter sheath assembly being connectable to a first carrier and a second catheter sheath component of the catheter sheath assembly being connectable to a second carrier; and
a control mechanism carried, in use, by a catheter handle and associated with the carriers to effect relative displacement between the carriers to cause displacement of a distal end of at least the first catheter sheath component between a non-deployed position and a deployed position.

2. The catheter actuator of claim 1 in which the first carrier and the second carrier are arranged to be displaceable relative to a further component of the catheter, at least a part of which is fast with a body of the catheter handle.

3. The catheter actuator of claim 2 in which the first carrier comprises a body to which the first catheter sheath component is connectable, the body defining a bore.

4. The catheter actuator of claim 3 in which the second carrier comprises a tubular member slidably received in the bore of the body of the first carrier with the second catheter sheath component being connectable to a distal end of the tubular member.

5. The catheter actuator of claim 4 in which the second carrier includes a follower which cooperates with the control mechanism for controlling relative displacement between the first carrier and the second carrier.

6. The catheter actuator of claim 5 in which the control mechanism includes a movement control member mounted, in use, fast with the catheter handle, the movement control member having a guide arrangement for guiding relative displacement between the first carrier and the second carrier.

7. The catheter actuator of claim 6 in which the control mechanism includes an actuator arm carried by the first carrier and which engages the movement control member, the actuator arm defining a receiving formation in which the follower of the second carrier is received.

8. The catheter actuator of claim 7 in which the actuator arm is pivotally mounted on the first carrier to be pivotable about a pivot axis.

9. The catheter actuator of claim 8 in which the actuator arm carries a cooperating formation which cooperates with the guide arrangement of the movement control member, the cooperating formation being arranged on one side of the pivot axis with the receiving formation being arranged on an opposed side of the pivot axis.

10. The catheter actuator of claim 9 in which the guide arrangement is a shaped guide slot defined in the movement control member, the cooperating formation of the actuator arm being a second follower received in the guide slot.

11. A catheter handle assembly comprising:
a handle body; and
a catheter actuator, as claimed in claim 10, carried by the handle body.

12. The catheter handle assembly of claim 11 in which the handle body defines a mounting arrangement for mounting the at least part of the further component of the catheter.

13. The catheter handle assembly of claim 12 in which a guide tube arrangement is carried within the handle body for guiding the further component.

14. The catheter handle assembly of claim 11 which includes a catheter sheath projection arrangement carried on a distal end of the handle body.

15. The catheter handle assembly of claim 14 in which the handle body is elongate and in which the catheter sheath projection arrangement is axially displaceably arranged on, the distal end of the handle body.

16. The catheter handle assembly of claim 14 in which the movement control member of the catheter actuator is arranged in a fixed position relative to the catheter sheath projection arrangement in a bore at the distal end of the handle body.

17. The catheter handle assembly of claim 16 in which the body of the first carrier of the catheter actuator is fast with the catheter sheath projection arrangement.

18. A catheter assembly comprises:
a catheter handle assembly as claimed in claim 11;
a stylet received in the handle body of the catheter handle assembly to project through a distal end of the handle body; and
a catheter sheath assembly mounted on, and extending distally from, the catheter handle assembly.

19. The catheter assembly of claim 18 in which the stylet is a steering shaft received in a lumen of the catheter sheath assembly.

20. The catheter assembly of claim 19 in which the steering shaft has a first member fixed within the handle body and a second member axially displaceably arranged relative to the first member.

21. The catheter assembly of claim 19 in which an electrode is carried on a distal end of the steering shaft.

22. The catheter assembly of claim 21 in which the catheter actuator is configured to displace the first catheter sheath component and the second catheter sheath component by a first amount relative to one another and the catheter sheath assembly, comprising the combination of the first catheter sheath component and the second catheter sheath component, by a second, greater amount relative to the steering shaft so that, when the first catheter sheath component is in its deployed position, the electrode of the steering shaft is operatively positioned relative to the deployed first catheter sheath component.

23. The catheter assembly of claim 22 in which, when the distal end of the at least first catheter sheath component is in its non-deployed configuration, the distal end of the at least first catheter sheath component lies proximally of the electrode of the steering shaft.
